(19) United States
(12) Patent Application Publication
(10) Pub. No.: US 2024/0083909 A1
(43) Pub. Date: Mar. 14, 2024

(54) 2D NON-CARBON NANOMATERIALS AND APPLICATIONS THEREOF

(71) Applicant: Olanrewaju Tanimola, Sugarland, TX (US)

(72) Inventor: Olanrewaju Tanimola, Sugarland, TX (US)

(21) Appl. No.: 17/931,113

(22) Filed: Sep. 9, 2022

(51) Int. Cl. C07D 495/16

(52) U.S. Cl. CPC C07D 495/16

(57) ABSTRACT

The present invention discloses methods for synthesis of 2D non-carbon nanomaterials or 2D complex oxides from materials with complex structure e.g. asphaltenes. It discloses a composition comprising a network of one or more 2D non-carbon nanomaterials having a lattice structure. The lattice structure is selected from an orthorhombic system, monoclinic system, triclinic system. The lattice structure is formed by (ASU)n, where ASU is asymmetric unit and n>=1, where ASU is (TxLyMz) where T is an alkaline metal, L is a chalcogen, O is oxygen, x>=5, y>=2, z>=8. The lattice structure is formed by (ASU)n, where ASU is asymmetric unit and n>=1, where ASU is (TxLyMz H) where T is an alkaline metal, L is a chalcogen, O is oxygen, x>=1, y>=1, z>=5. Examples of the disclosed composition are a network of one or more 2D nanostructures having an asymmetric unit of NaSO₅H or Na₂S₂O₈.

### CROSS-REFERENCE TO RELATED APPLICATIONS

This application claims the benefit of the filing date of and priority to: U.S. Provisional Application Ser. No. 62/035,140 entitled “Methods for Synthesis of Graphene Derivatives and Functional Materials from Asphaltenes”, filed Aug. 10, 2015, Confirmation No. 9603; the entire contents of the said provisional application is hereby incorporated by reference.

### STATEMENT REGARDING FEDERALLY SPONSORED RESEARCH OR DEVELOPMENT

Not Applicable.

### BACKGROUND OF THE INVENTION

Two dimensional (2D) non-carbon nanomaterials are emerging nanostructured material with suitable capabilities needed for the advancement of numerous technologies. Several 2D non-carbon materials have emerged in the past few years. These include as black phosphorus (BP or phosphorene), transition metal oxides (TMOs), layered double hydroxides (LDHs), transition metal carbides and carbonitrides (MXenes), and covalent organic frameworks (COFs). As well as 2D oxides such as layered CuO, MoO3 and WO3. Properties such as, folded structure, high electrical conductivity, and excellent flexibility, pave the way to an exciting range of novel applications like catalysis, electronics, energy conversion and storage, sensing, photonics, nanocomposites, and membranes. The selectivity of this class of material in a wide range of application is attributed to tunable band gap property, which can be altered through material processing, for instance, by changing the number of layers.

Similar to carbon nanostructures, non-carbon 2D materials can be produced through processes like chemical exfoliation, chemical vapor deposition (CVD), solution phase growth. However, progress in the advancement of this family of 2D nanostructures is hindered by limitations such a by the aggregation of 2D materials in solvents, dispersibility and processability concerns, scalability, and environmental stability issues. Chemical functionalization is often utilized for preserving their large aspect ratio and high specific surface area, enhance processability, improve physicochemical properties and environmental stability, while imparting new properties. Chemical functionalization can be categorized into non-covalent and covalent functionalization. Non-covalent interactions explore electrostatic, π-π, or hydrophobic interactions to physically adsorbed organic units onto 2D materials. It is often used to preserve the structural and intrinsic properties of 2D materials. On the other hand, covalent interactions promote direct bond linkage of organic moieties with 2D materials through cycloaddition, condensation, radical addition reactions etc. A cost-effective strategy that enable structural control, reproducibility and scalability of chemical functionalization could be impactful for non-carbon 2D technologies.

An object of this invention is to develop a more economic, scalable, and sustainable way of producing 2D non-carbon nanostructures.

An object of this invention is to develop a tunable and novel 2D non-carbon nanostructures and applications thereof.

An object of this invention is to develop a composition comprising tunable and novel 2D non-carbon nanostructures and applications thereof. The application comprises electrodes, automobiles, biomedical, catalysis, lighting, optics, energy conversion and storage, sensing, photonics, nanocomposites, and membranes

Moreover, the impact of CO2 on climate change has ignited new development in materials processing. In recent years, tremendous efforts are being committed towards developing synthetic approach that captures and utilize CO2 in a sustainable manner.

There is a need for a climate change mediation approach which could be in the form of CO2 usage.

Certain aspects of the present invention relate to process steps that include the use of CO₂ as an additive. The process steps relate to contacting an asphaltene composition with CO₂ to form a feedstock of present invention.

### SUMMARY OF THE INVENTION

The present invention discloses methods for synthesis of 2D non-carbon nanomaterials from materials with complex structure e.g. asphaltenes.

The present invention discloses methods for synthesis of 2D complex oxides from materials with complex structure e.g. asphaltenes.

The present invention discloses methods for synthesis of reduced 2D complex oxides from materials with complex structure e.g. asphaltenes.

The present invention discloses a composition comprising a network of one or more 2D non-carbon nanomaterials having a lattice structure. The lattice structure is selected from an orthorhombic system, monoclinic system, triclinic system.

The lattice structure is formed by (ASU)n, where ASU is asymmetric unit and n>=1, where ASU is (TxLyMz) where T is an alkaline metal, L is a chalcogen, O is oxygen, x>=5, y>=2, z>=8.

The lattice structure is formed by (ASU)n, where ASU is asymmetric unit and n>=1, where ASU is (TxLyMz H) where T is an alkaline metal, L is a chalcogen, O is oxygen, x>=1, y>=1, z>=5.

The present invention discloses a composition comprising a network of one or more 2D nanostructures having an asymmetric unit of NaSO₅H.

The present invention discloses a composition comprising a network of one or more 2D nanostructures having an asymmetric unit of Na₂S₂O₈

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 shows single X-ray crystallography of a network of one or more 2D nanostructures having an asymmetric unit of Na₂S₂O₈ of present invention.

FIG. 2 shows single X-ray crystallography of a network of one or more 2D nanostructures having an asymmetric unit of NaSO₅H of present invention

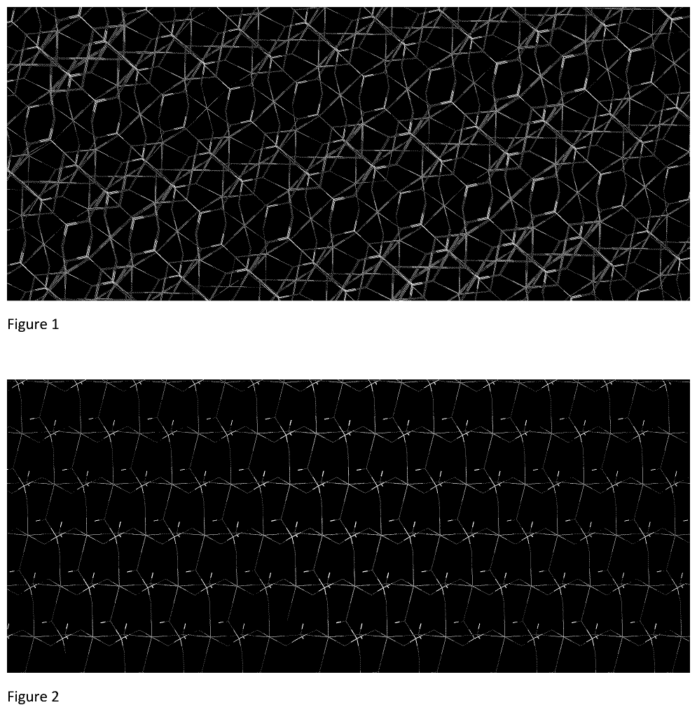

### DETAILED DESCRIPTION OF THE INVENTION

Asphaltene is a complex mixture comprising many components which indeed may vary from one crude oil to the other. Up to date, the exact composition and molecular structure of asphaltenes remain unresolved. Asphaltenes represent a chemically complex and structurally heterogeneous group of organic molecules that are present in crude oil.

Asphaltene compounds are involved in the precipitation of organic deposits in petroleum reservoirs, the formation and stabilization of emulsions, and the wettability of transport pipelines during transportation. Wetting is the ability of a liquid to maintain contact with a solid surface, resulting from intermolecular interactions when the two are brought together. The degree of wetting or wettability is determined by a balance between adhesive and cohesive forces. Wetting is important in the bonding or adherence of two materials. As a result of its adsorption capability, asphaltene has the propensity to form colloidal aggregations in solution thereby promoting stable water in oil emulsions. These characteristics of asphaltene are problematic in oil production and result in large maintenance costs.

Certain embodiments are directed to methods and compositions for the production or synthesis of materials from asphaltenes (making asphaltene derivatives). Modification or functionalization methods facilitate the synthesis of asphaltene derivatives that vary in morphology, size, shape, bonding, absorption, transmission, fluorescence, color, supramolecular structure, lattice structure, diffraction pattern, functional groups, etc.

Asphaltenes can be functionalized and the properties of asphaltene derivatives manipulated by altering the conditions, reagents, and parameters of the methods described herein. In one embodiment this method may be regarded as a functionalization, oxidation, and reduction process. In certain aspect the reaction involves one or more procedures that involves physisorption and chemisorption at an asphaltene-solvent interface, which facilitates conjugation of a fragment of asphaltenes with an active component of the solvent. The reaction may result in the functional groups being absorbed either on the surface of the asphaltene aggregation or become bonded with the polar group of asphaltene molecules to form functionalized asphaltene. Modification of the surface may result in the elimination or substitution of the functional group with a substituent group.

Various groups have reported on the characterization of asphaltene structure, yet consensus remains elusive. Two primary model structures have been proposed. One includes the island model which depicts one poly-aromatic hydrocarbon (PAH) architecture and the second describes it as a multiple cross-linked PAHs architecture (the archipelago architecture) (Strausz et al., 1992, Fuel, 71, 1355-1363; Groenzin and Mullins, 2000, Fuels, 14 (3), 677-684; Gray, 2003, Energy and Fuels, 17 (6), 1566-1569). Despite this controversy over structure, the current description provides methods for producing a variety of products using asphaltenes as a source material.

A. Asphaltene Derivatization

Asphaltenes are typically characterized as macromolecules with rigid core aromatic rings and flexible alkyl chains with a variable number of heteroatoms and metals (Andersen and Speight, 1999, Jour. Pet. Sci. Eng., 22, 53-60). Asphaltenes typically contain oxygen (0.3-4.8%), sulfur (0.3-10.3%), nitrogen (0.6-3.3%), and small amounts of metals, such as Fe, Ni, and V (Buch et al., 2003, Fuel, vol. 82 (9), 1075-1084; Groenzin and Mullins, 2000, Fuels, 14 (3), 677-684).

The presence of electronegative heteroatoms like nitrogen or oxygen in the structure of asphaltene results in an asphaltene being a strong hydrogen bond acceptor and weak hydrogen bond donor, which allows for the use of a dispersant when working with asphaltenes. Strong acids become effective asphaltene dispersants if their alkyl tails are long enough to provide the necessary steric-stabilization layers around the asphaltenes. Besides, organic salts may be strong donors of the hydrogen bonding and may also instill stability on asphaltenes structure. It is apparent that the pH of a solution is a determining factor. In another embodiment long alkyl chain compounds are employed for the dispersion of asphaltene.

In certain aspects asphaltenes are reacted with an oxidizing acid. An oxidizing acid is a Brønsted acid that is also a strong oxidizing agent. All Brønsted acids can act as oxidizing agents, because the acidic proton can be reduced to hydrogen gas. Some acids contain other structures that act as stronger oxidizing agents than hydrogen ion. They contain oxygen in the anionic structure. These include, but are not limited to nitric acid, perchloric acid, chloric acid, chromic acid, and sulfuric acids.

Asphaltene can be derivatized by contacting with one or more oxidants.

Asphaltene can be derivatized by refluxing an asphaltene and one or more surfactants.

Asphaltene can be derivatized by refluxing an asphaltene and one or more dispersants.

Asphaltene can be derivatized by performing chemical one or more reduction steps on a dispersed asphaltene composition or a refluxed asphaltene composition.

It is an object of the present invention to provide a method for synthesizing 2D non-carbon nanomaterial from asphaltenes that includes an asymmetric unit provides precise details of the atoms and molecules that make up the building block of layered nanostructure. In addition, present invention discloses method of controlling number of layers or isolating individual atomic planes from a bulk composition like asphaltene.

It is an object of the present invention to provide a method for synthesizing 2D non-carbon nanomaterial from asphaltenes that discloses a reproducible and scalable production process of transforming asphaltene into 2D non-carbon materials with control over number of layers, size, and morphology.

The surfactant comprises a sulfonic acid composition or alkyl sulfonic acid oxonium salt.

The dispersant comprises a selection from p-alkylphenols, p-alkylbenzenesulfonic acid, or alkyl sulfonic acid.

Prior art described surfactants; WO 2013/165869 is incorporated herein for all purposes of the processing.

Similarly, prior arts disclosed a large number of reductants such as: hydrogen sulfide, sodium borohydride, hydrazine, an alkaline solution, sodium citrate, amino acids, sugars, ascorbic acid, thermal energy, electromagnetism, polyphenols, tea, and microorganisms have been reported

This method is a recycling method that can further comprise derivatising asphaltene. In one embodiment, the starting material comprises any of: layered materials, stacked materials, asphaltenes, two dimensional materials, molybdenum sulfide, boron nitride, carbon slurry, graphitic compounds, soot, lignite, peat, layered materials, PAH compounds, resin, graphite, petrified oil, asphalt, bitumen, modified bitumen, coal, modified coal, modified asphaltenes, anthracite, modified anthracite and combinations thereof.

B. Methods for Making 2D Non-Carbon Nanomaterials

Generally, the method comprises: providing an asphaltene composition, performing one or more processing steps selected from: refluxing the asphaltene composition with one or more additives to form a refluxed asphaltene derivative, contacting the refluxed asphaltene derivative with one or more reducing agents to form a reduced asphaltene derivative; purifying the refluxed asphaltene derivative or the reduced asphaltene derivative.

The asphaltene composition comprises an asphaltene material and one or more additives.

The asphaltene composition comprises an asphaltene material, one or more solvents and one or more surfactants.

The asphaltene composition comprises an asphaltene material, one or more organic solvents and one or more dispersants.

The asphaltene composition comprises an asphaltene material, one or more organic solvents and one or more oxidants.

The oxidant comprises a selection from mixed acid, hydrogen peroxide, methanol, ammonia, carbonic acid, hydrochloric acid, taurine, nitric acids, sulfuric acids, water, alkyl toluene sulfonic acid and any combination thereof.

The solvent comprises a selection from ammonia, methanol, aromatic solvents, water, tetrahydrofuran, diethyl ether, carbon tetrachloride, hydrogen peroxide, sulfate, alkyl phenol, toluene, benzene, xylene and any combination thereof.

The surfactant comprises a selection from alkyl sulfonic salt, alkyl sulfonic acid oxonium salt. non-ionic, anionic, cationic, amphoteric surfactants and zwitterionic surfactants, janus surfactants, and mixtures thereof.

The dispersant comprises a selection from p-alkylphenols, p-alkylbenzene sulfonic acid, or alkyl sulfonic acid.

The method further comprises purifying steps including filtration, centrifugation, dialysis, solvent extraction, recrystallization, and any combinations thereof.

The method contacting the refluxed asphaltene derivative with one or more modifying agents to form a modified asphaltene derivative.

The modifying agent may comprise any of sodium hydroxide, sodium borohydride, methanol, dioxane, hydrochloric acid, sodium tetrahydrofuran, carbon tetrachloride, copper chloride, phosphoric acid, water, methanol, hydrochloric acid, sodium nitrite, sodium sulfide, copper bromide and a combination thereof.

The method can further comprise separating modified solution into carbon powder and continuous phase using any of filtration, centrifugation, dialysis, washing, recrystallization, solvent extraction, addition of solvent, and a combination thereof.

The method can further comprise contacting the modified asphaltene derivative with one or more controlling agents to form a controlled asphaltene derivative.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description

The starting material may include crude asphaltene or treated asphaltenes e.g., thermally treated asphaltenes, milled asphaltene, mechanically treated, thermo-mechanically treated asphaltene as starting material.

The term “additive” include of heteroatom containing surfactants, acids, mixed acids, solvents, organic solvents, antifoulants, coagulants, flocculants, solubilizing agents, antifoaming agent, emulsifying agents, dispersing agents, ionic liquids, salts, oxidizing agents, reducing agents, catalysts, amphiphilic solvents, dispersants, oxidants, acids, polymer copolymer, biological macromolecules, acidic solution, DNA, protein molecules, organic solvents, polar solvents, non-polar solvents, aromatic solvents, water, alcohol, alkane, acidic ionic liquids and combinations thereof.

In certain aspects the intercalating agents or additives may be selected from any of but not limited to nitric acid, sulfuric acid, aromatic solvent, polar solvent, methanol, water, peroxide, formic acid, benzene, toluene, naphthalene, nitrobenzene sulfonic acid, linear alkyl sulfonic, polytetrafluoroethylene, sodium nitro sulfonic acid, sodium dodecyl sulfate, cetyltrimethylammoniumbromide, phospholipid, lignin, sulfuric acids, ammonia, taurine, tetrahydrofuran, sulfur trioxide, carboxylic acid, acetic acid, carboxylic acids, sulfonic acids and derivatives, carbonic acids, nitro-sulfonic acids, oleic acid, methyl cellulose, diethylene glycol, polyoxyethylene sorbitan monolaurate, hydrogen peroxide, oxalic acid, perchloric acid, iron bromide, iron chloride, phosphoric acid, hydrofluoric acid, chlorosulfonic acid, trifluoromethanesulfonic acid, nickel, iron, vanadium, permanganate, oleum, chloride, chlorite, nitrite, potassium permanganate, methanol, acetone, water, propanol, isopropanol, dimethyl sulfoxide, polyethylene glycol (PEG), polymer copolymer (PEtOz-Pcl), alkyl phenol, water, Dodecyl benzenesulphonic acid, methanol, tetrachloromethane, taurine, methionine, trichloromethane, tetrahydrofuran, N-(3-dimethylaminopropyl), N′ethyl-carbodiimide hydrochloride, N′ hydroxyl succinimide, poly-lysines (PLs), sodium dodecyl benzenesulfonate, platinum, sodium oleate, cysteine, homocysteine, or mixtures thereof.

A co-agent or modifying agent refers to a solvent that contributes to the tuning of the surface or interphase of the refluxed asphaltene solution. A modifier is introduced to alter the edges of the asphaltene emulsion. Such action may result in the change in the structural dimension. In certain aspect a modifier performs several operations that include without limitation addition of functional group(s), elimination of the functional group(s), and substitution of functional group(s).

The modifying agents comprise without limitation metal catalyst, basic solution, a reducing agent, a salt solution, metal oxides, sulfides, hydrides and any combination thereof. In certain aspects a modifier is selected from any of sodium chloride, sodium hydroxide, ammonium hydroxide, hydrochloric acid, tetrahydrofuran, amino triazole, dimethylformamide, dimethylsulfoxide, phosphines, phosphites, sulfites, sulfides, hydrosulfites, borohydrides, boranes, hydroxylamine, lithium alhydride, sodium nitrite, hydrochloric acid, copper bromide, copper cyanide, potassium iodide, borohydrides, hydrochloric acid, cyanoborohydrides, aluminum hydrides, hydroquininone, hydrogen dimethylhydrazine, N,N-dimethylhydroxylamine, methylamine, dioxane, amino acids, dimethylamine, trimethylamine taurine, methionine, potassium hydroxide, tin, methanol, alkali sulfide, distilled water, or mixtures thereof.

A controlling agent is added to the solution when there is a need to neutralize the reaction in order to obtain a stable composition. The controlling agent may also modify the pH of the solution. The selection of a controlling agent is dependent on the pH of the solution; the controlling agent is selected from any of hydrochloric acid, sodium bicarbonate, methanol, dimethyl sulfoxide, sodium hydroxide, potassium hydroxide, tetrahydrofuran, dimethylformamide, alkali sulfide, dimethylsulfoxide, phosphines, phosphites, sulfites, sulfides, dioxane, hydrosulfites, borohydrides, boranes, distilled water, sodium bicarbonate or mixtures thereof.

In certain aspects surfactants, sulfonic derivatives, mixed acids, nitric acid, sulfuric acid, sodium hydroxide, and hydrochloric acid can be used in the methods described. However, other reagents with similar characteristics can also be used in place of these particular reagents.

Certain aspect of present invention relates to the use of solvent selected from the group consisting of organic solvents, polar, polar aprotic, apolar, aromatic, and a mixture thereof. In certain aspects the solvent is selected from benzene, acetonitrile, heptane, hexane, propane, water, toluene, naphthalene, methanol, distilled water, dichloromethane, chloroform, xylene, dichlorobenzene, dimethylformide, tetrahydrofuran, chlorobenzene, dioxane, ethyl glycerol, alkanes, methylbenzene, ethyl benzene, isopropyl benzene, methyl naphthalene, dimethysulfoxide, isopropanol, tetrahydrofuran, dimethylformamide, carbon sulfide, 1,2 dichloromethane, acetone, tetrachloromethane, sulfur trioxide, chloroform, dichlorobenzene, and combinations thereof. result in the elimination or substitution of the functional group with a substituent group.

In certain aspects the intercalating solvent (surfactant) is an oxidative acid or a mixture of oxidative acids. An oxidative acid can be sulfuric acid, perchloric acid, nitric acid, or mixtures thereof. An acid component can be present at final concentration of 0.0001, 0.0005, 0.001, 0.005, 0.01, 0.05, 0.1, 0.2, 0.3, 0.4, 0.5, 0.6 M to 0.5, 0.6, 0.7, 0.8, 0.9, 1 M during reflux. An asphaltene/solvent/oxidative acid mixture can be refluxed at a temperature of 25, 30, 35, 40, 45, 50, 55, 60, 65, 70, 75, 80, 85, 90, 95, 100□C or greater, including all values and ranges there between. The pH of the solution, which during reflux is less than 2 can be controlled by neutralizing the mixture with a base, such as diluted NaOH, KOH, or the like (0.0001 M to 1M). Quenching can be accomplished by addition of an acidic solution (e.g., HCl) (0.0001 M to 1M). Varying the parameters results in variation of the attribute of the resulting product (e.g., structural morphology, lattice parameter, degree of orderliness etc.). Therefore, all variables (e.g., solvents, temperature range, concentration, volume, ratio, time etc.) can be varied to some degree to alter the characteristics of the products.

The 2D non-carbon nanomaterial of present invention is formed by contacting an asphaltene derivative with one reducing agents or reductants.

The reducing agent or reductant is selected from hydrogen sulphide, sodium borohydride, hydrazine, an alkaline solution, sodium citrate, amino acids, sugars, ascorbic acid, thermal energy, electromagnetism, polyphenols, tea, and microorganisms.

Present invention relates to a method of producing 2D non-carbon nanomaterial by contacting an asphaltene derivative with one or more reducing agents or reductants selected from hydrogen sulphide, sodium borohydride, hydrazine, an alkaline solution, sodium citrate, amino acids, sugars, ascorbic acid, thermal energy, electromagnetism, polyphenols, tea, and microorganisms

As stated in known arts, the present invention incorporates the use of vapor deposition to enhance the quality of the disclosed 2D non-carbon nanomaterial process. This includes chemical vapor deposition, thermal vapor deposition, etc.

I claim:

1. A composition comprising:
a network of one or more 2D non-carbon nanomaterials having a lattice structure.
The lattice structure is selected from an orthorhombic system, monoclinic system, triclinic system.
The lattice structure formed by (ASU)n, where ASU is asymmetric unit and n>=1, where ASU is (TxLyMz)n where T is an alkaline metal, L is a chalcogen, O is oxygen, x>=5, y>=2, z>=8.
The lattice structure formed by (ASU)n, where ASU is asymmetric unit and n>=1, where ASU is TxLyMz H where T is an alkaline metal, L is a chalcogen, O is oxygen, x>=1, y>=1, z>=5.

2. The lattice structure according to claim 1 further comprising an asymmetric unit (Na₂S₂O₈) of present invention.

3. The lattice structure according to claim 1 further comprising a network of one or more 2D nanostructures having an asymmetric unit of NaSO₅H

4. A method for synthesis of non-carbon nanomaterial comprising:
providing an asphaltene composition, performing one or more processing steps selected from:
refluxing the asphaltene composition with one or more additives to form a refluxed asphaltene derivative, contacting the refluxed asphaltene derivative with one or more reducing agents to form a reduced asphaltene derivative; purifying the refluxed asphaltene derivative or the reduced asphaltene derivative.

5. The asphaltene composition according to claim 4 further comprises an asphaltene material and one or more additives.

6. The asphaltene composition according to claim 4 further comprises an asphaltene material, one or more solvents and one or more surfactants.

7. The asphaltene composition according to claim 4 further comprises an asphaltene material, one or more organic solvents and one or more dispersants.

8. The asphaltene composition according to claim 4 further comprises an asphaltene material, one or more organic solvents and one or more oxidants.

9. The oxidant according to claim 8 further comprises a selection from mixed acid, hydrogen peroxide, methanol, ammonia, carbonic acid, hydrochloric acid, taurine, nitric acids, sulfuric acids, water, alkyl toluene sulfonic acid and any combination thereof.

10. The solvent according to previous claims further comprises a selection from ammonia, methanol, aromatic solvents, water, tetrahydrofuran, diethyl ether, carbon tetrachloride, hydrogen peroxide, sulfate, alkyl phenol, toluene, benzene, xylene and any combination thereof.

11. The surfactant according to claim 6 further comprises a selection from alkyl sulfonic salt, alkyl sulfonic acid oxonium salt. non-ionic, anionic, cationic, amphoteric surfactants and zwitterionic surfactants, janus surfactants, and mixtures thereof.

12. The dispersant according to claim 7 further comprises a selection from p-alkylphenols, p-alkylbenzene sulfonic acid, or alkyl sulfonic acid.

13. The method according to claim 4 further comprises purifying steps including filtration, centrifugation, dialysis, solvent extraction, recrystallization, and any combinations thereof.

14. The method according to claim 4 further comprises contacting the refluxed asphaltene derivative with one or more modifying agents to form a modified asphaltene derivative.

15. The modifying agent according to claim 4 further comprises a selection from sodium hydroxide, sodium borohydride, methanol, dioxane, hydrochloric acid, sodium tetrahydrofuran, carbon tetrachloride, copper chloride, phosphoric acid, water, methanol, hydrochloric acid, sodium nitrite, sodium sulfide, copper bromide and a combination thereof.

16. The method according to claim 4 further comprises performing one or more purification techniques selected from filtration, centrifugation, dialysis, washing, recrystallization, solvent extraction, and a combination thereof.

17. The method according to claim 4 further comprises performing vapor deposition technique
